(19) United States
(12) Patent Application Publication
(10) Pub. No.: US 2019/0146392 A1
(43) Pub. Date: May 16, 2019

(54) SENSOR DEVICE AND IMAGE FORMING APPARATUS

(71) Applicant: SHARP KABUSHIKI KAISHA, Sakai City (JP)

(72) Inventor: YUSUKE SAKAKIBARA, Sakai City (JP)

(21) Appl. No.: 16/147,585

(22) Filed: Sep. 29, 2018

(30) Foreign Application Priority Data
Nov. 14, 2017 (JP) 2017-219262

(51) Int. Cl. G03G 15/00; G01B 11/06; B41J 11/00; G01N 21/17; G01N 33/34

(52) U.S. Cl. CPC G03G 15/5029; G01B 11/06; B41J 11/009; G03G 2215/00738; G01N 33/346; G01N 2201/06113; G03G 2215/00734; G01N 21/17

(57) ABSTRACT

A sensor device includes a light emitter, a photodetector configured to receive, during a first state where no paper exists, irradiated light from the light emitter, and to receive, during a second state where a paper exists, transmitted light from the paper, an amplifier circuit configured to convert an output of the photodetector into a voltage, a storage circuit configured to hold an output voltage, during the first state, from the amplifier circuit, and an AD converter configured to set an output voltage of the storage circuit as a reference voltage, and to convert, into a digital value, a voltage ratio between the reference voltage and an output voltage, during the second state, of the amplifier circuit. Based on an output from the AD converter, a paper thickness is detected.

### CROSS-REFERENCE TO RELATED APPLICATIONS

This application claims the benefit of priority to Japanese Patent Application No. 2017-219262 filed on Nov. 14, 2017. The entire contents of the above-identified application are hereby incorporated by reference.

### BACKGROUND

### Technical Field

The present disclosure relates to a sensor device configured to detect paper characteristics and provided in an image forming apparatus configured to form an image onto a paper, and the image forming apparatus.

In an image forming apparatus, such as a copier, a printer, a facsimile, and a multifunctional peripheral of the copier, the printer, and the facsimile, various kinds of papers are used, such as pure paper, recycled paper, thin paper, thick paper, and coated paper. To improve quality of an image formed by the image forming apparatus, image forming conditions, such as transfer current, fusing pressure, fusing temperature, and fusing time, should be set in accordance with a thickness of a paper (basis weight). For this purpose, an image forming apparatus equipped with a sensor configured to detect a thickness of a paper (basis weight) has been developed.

For example, JP 2009-8808 A (published on Jan. 15, 2009) describes an image forming apparatus configured to measure transmittance of a paper to determine a paper type. That is, as illustrated in FIG. 16, in a state where no paper exists (first, state), a photodetector 104 receives light. An amplifier circuit 116 then performs amplification. An analog voltage V0 is thus output. Next, an analog-digital (AD) converter 113 compares the analog voltage V0 with a constant reference voltage Vref. A digital value D0 corresponding to a voltage ratio V0/Vref is thus output. The digital value D0 is stored in a storage 112a, for example. Next, in a state where a paper exists (second state), transmitted light from the paper is received. Similarly, conversion then takes place to obtain a digital value D1 corresponding to a voltage ratio V1/Vref between an analog voltage V1 and the reference voltage Vref. The digital value D1 is thus stored. Finally, a calculator 112b, for example, calculates a digital ratio D1/D0 between digital signals. The digital ratio is then regarded as transmittance of the paper. A paper type is thus determined.

### SUMMARY

However, when the digital ratio D1/D0 is smaller (approximately 0.1 or less), measuring the digital ratio D1/D0 at higher precision has been difficult.

In the AD converter 113, digital conversion takes place as described below. For example, a 10-bit AD converter outputs a digital value (0 to 1023) representing a rank of a magnitude of a voltage, within a range from 0 to Vref inclusive divided into 1024 (=2¹⁰). For a voltage having digital value exceeding 1023, an over range event occurs, disallowing measurement. A fractional portion of a digital value will be rounded down. This means that a digital value to be output includes an error of approximately 1 (quantizing error).

To measure the digital ratio D1/D0 at a precision of 1%, it is required that both the digital value D0 and the digital value D1 be measured at a precision of 1%. By increasing an amplification factor of the amplifier circuit 116, the digital value D0 and the digital value D1 increase, relatively reducing a quantizing error through digitalization. On the other hand, to avoid an over range event, an amplification factor is set to reduce the digital value D0. In general, an amplification factor of approximately 500 is set for the digital value D0 to avoid an over range event even when taking into account factors including fluctuation of an amount of light being emitted from a light emitter 103, individual differences due to procurement in a greater quantity for mass production, fluctuation and an individual difference in sensitivity of the photodetector 104, and fluctuation and an individual difference in amplification factor of the amplifier circuit 116, for example. When the digital ratio D1/D0 is 0.1 or less, the digital value D1 is 50 or less, leading to another issue that, due to a quantizing error through digitalization, precision in measurement of the digital value D1 lowers below 2%. Increasing the AD converter 113 in bit count leads to still another issue, i.e., an increase in cost.

In view of the issues described above, the present disclosure has, in an aspect, an object to simplify a circuit configuration, and to achieve a sensor device and an image forming apparatus capable of determining paper characteristics at higher precision, and of forming a high quality image.

To solve the issues described above, the sensor device according to an aspect of the present disclosure includes a light emitter configured to irradiate light, a photodetector configured to receive, during a first state where no paper exists, irradiated light from the light emitter, and to receive, during a second state where a paper exists, transmitted light from the paper, an amplifier circuit configured to convert an output of the photodetector into a voltage, a storage circuit configured to hold an output voltage, during the first state, from the amplifier circuit, and an AD converter configured to set an output voltage of the storage circuit as a reference voltage, and to convert, into a digital value, a voltage ratio between the reference voltage and an output voltage, during the second state, of the amplifier circuit. Based on an output from the AD converter, paper characteristics are detected.

To solve the issues described above, a sensor device according to an aspect of the present disclosure includes a light emitter configured to irradiate light, a first photodetector configured to receive irradiated light from the light emitter, a second photodetector configured to receive transmitted light from a paper, among the irradiated light from the light emitter, a first amplifier circuit configured to convert an output of the first photodetector into a voltage, a second amplifier circuit configured to convert an output of the second photodetector into a voltage, and an AD converter configured to set an output voltage of the first amplifier circuit as a reference voltage, and to convert, into a digital value, a voltage ratio between the reference voltage and an output voltage of the second amplifier circuit. Based on an output from the AD converter, paper characteristics are detected.

To solve the issues described above, an image forming apparatus according to an aspect of the present disclosure includes one of the sensor devices described above. An image forming condition is set based on a result of measurement by the sensor device.

The aspects of the present disclosure simplify a circuit configuration, and achieve a sensor device and an image forming apparatus capable of determining paper characteristics at higher precision, and of forming a high quality image.

### BRIEF DESCRIPTION OF DRAWINGS

The disclosure will be described with reference to the accompanying drawings, wherein like numbers reference like elements.

FIG. 1 is a block diagram illustrating a configuration of a main part of a sensor device according to a first embodiment.

FIG. 2A is a view illustrating a first state of a sensor of the sensor device. FIG. 2B is a view illustrating a second state of the sensor of the sensor device.

FIG. 3 is a circuit diagram illustrating a configuration of the main part of the sensor device.

FIG. 4 is a flowchart illustrating processing performed by an image forming apparatus including the sensor device.

FIG. 5 is a block diagram illustrating a configuration of a main part of a sensor device according to a second embodiment.

FIG. 6 is a circuit diagram illustrating a configuration of the main part of the sensor device.

FIG. 7 is a graph illustrating outputs of an amplifier circuit and a storage circuit in the sensor device.

FIG. 8 is a flowchart illustrating processing performed by an image forming apparatus including the sensor device.

FIG. 9A is a view illustrating the first state of a sensor of a sensor device according to a third embodiment. FIG. 9B is a view Illustrating the second state of the sensor of the sensor device.

FIG. 10 is a block diagram illustrating a configuration of a main part of the sensor device.

FIG. 11 is a flowchart illustrating processing performed by an image forming apparatus including the sensor device.

FIG. 12A is a view illustrating the first state of a sensor of an image forming apparatus according to a modification to the third embodiment. FIG. 12B is a view illustrating the second state of the sensor of the image forming apparatus according to the modification.

FIG. 13A is a view illustrating the first state of a sensor of an image forming apparatus according to a fourth embodiment. FIG. 13B is a view illustrating the second state of the sensor of the image forming apparatus.

FIG. 14 is a block diagram illustrating a configuration of a main part of the image forming apparatus.

FIG. 15 is a flowchart illustrating processing performed by the image forming apparatus including a sensor device.

FIG. 16 is a block diagram illustrating a configuration of a main part; of an ordinary image forming apparatus.

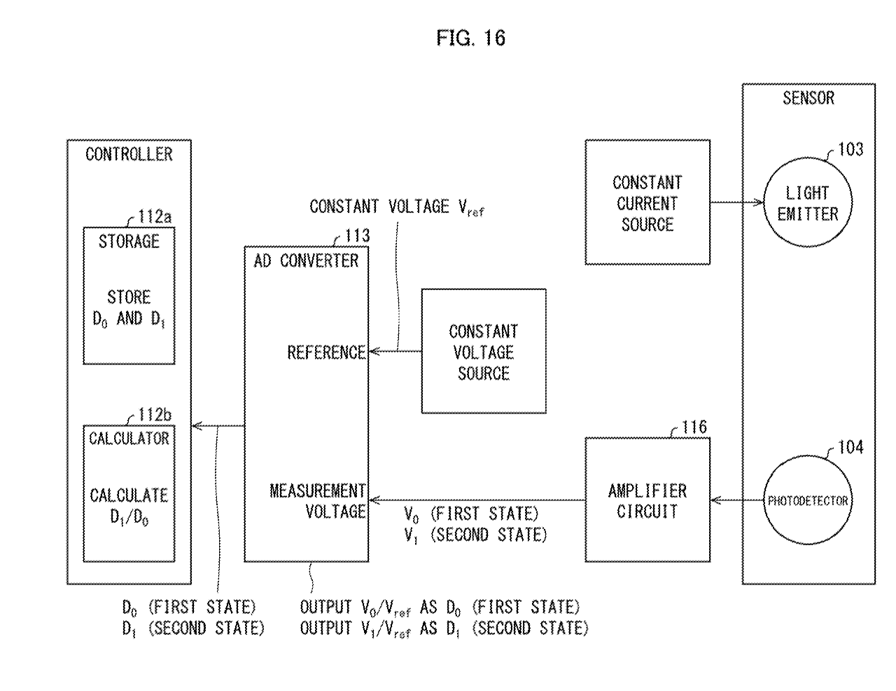

### DESCRIPTION OF EMBODIMENTS

### First Embodiment

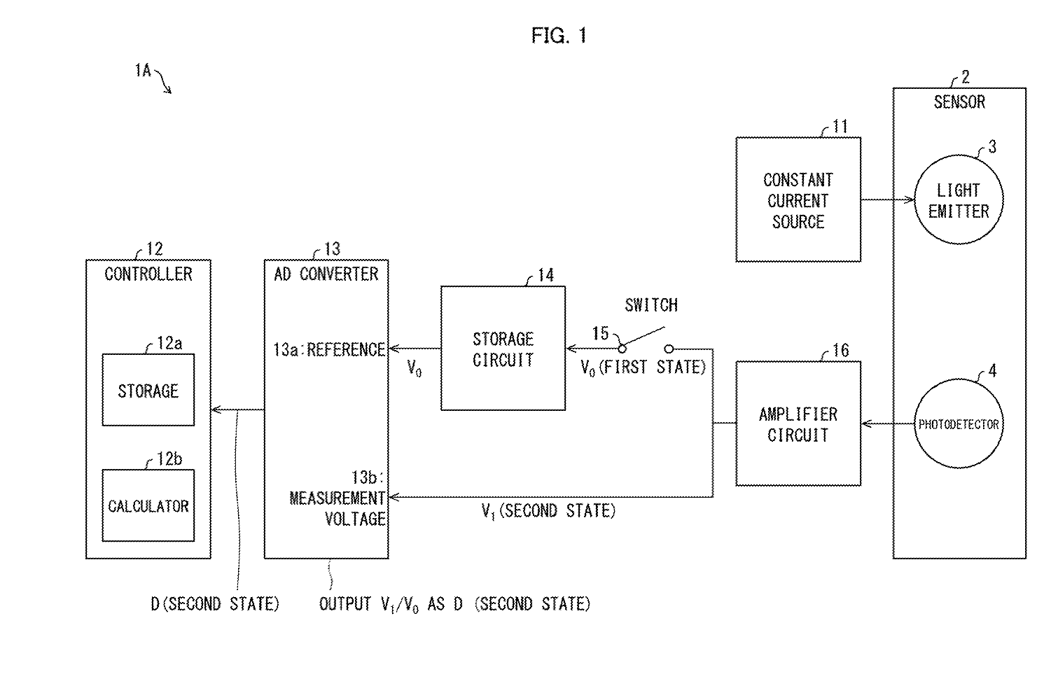

One embodiment of the present disclosure will be described with reference to FIGS. 1 to 4. The present embodiment describes a sensor device and an image forming apparatus, such as a copier, a printer, a facsimile, and a multifunctional peripheral of the copier, the printer, and the facsimile. The sensor device is useful for the image forming apparatus, and has a simplified circuit configuration. The sensor device and the image forming apparatus are configured to promptly detect a paper thickness (basis weight), and to set a printing condition.

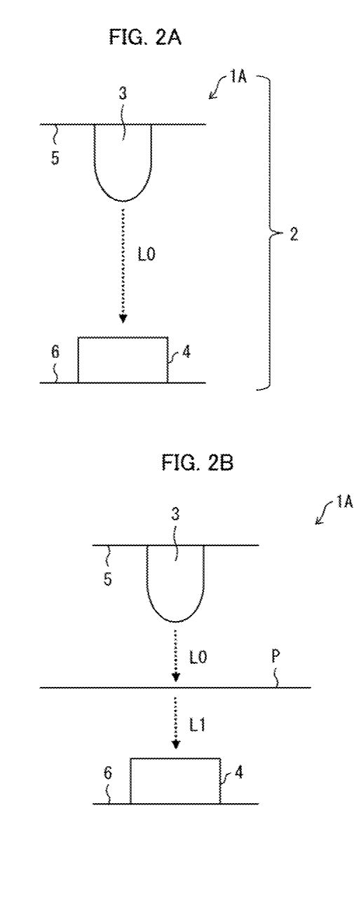

FIG. 2A is a view illustrating a first state of a sensor 2 of a sensor device 1A according to the present embodiment. The first state is referred to as a state where no paper P exists. FIG. 2B is a view of a state when the paper is fed to the sensor 2 (i.e., a second state, the paper P is either being fed or stationary while being fed, for example).

In the image forming apparatus configured to form an image onto the paper P, the sensor 2 is attached on a paper feeding path used to feed the paper P, for example.

As illustrated in FIG. 2A, the sensor 2 of the sensor device 1A includes a light emitter 3, a photodetector 4, and substrates 5 and 6 respectively attached with the light emitter 3 and the photodetector 4. The light emitter 3 is configured to irradiate irradiated light L0. During the first state, the irradiated light L0 enters the photodetector 4. On the other hand, as illustrated in FIG. 2B, during the second state, the irradiated light L0 is absorbed by the paper P, and scatters and enters into the photodetector 4 as transmitted light L1.

In the present embodiment, the light emitter 3 is a light emitting diode (LED). The light emitter 3 may be another light source than the LED, such as a laser light source.

During the first state, the photodetector 4 receives the irradiated light L0 irradiated from the light emitter 3. During the second state, the photodetector 4 receives the transmitted light L1 transmitted through the paper P. The photodetector 4 is a photodetecting sensor, and is a photodiode in the present embodiment. The present disclosure is not limited to the example described above. The photodetector 4 may be a phototransistor or a photo integrated circuit (IC), for example.

The substrates 5 and 6 are substrates respectively attached with the light emitter 3 and the photodetector 4.

The paper P is a paper to be formed with an image by the image forming apparatus. The paper P may be a pure paper, a recycled paper, a thin paper, a thick paper, or a coated paper, for example.

FIG. 1 is a block diagram illustrating a configuration of a main part of the sensor device 1A. As illustrated in FIG. 1, the sensor device 1A includes the sensor 2, a constant current source 11, a controller 12, an analog-digital (AD) converter 13, a storage circuit 14, a switch 15, and an amplifier circuit 16. The sensor 2 is already described with reference to FIG. 2A, and thus is not described repeatedly.

The constant current source 11 is configured to output a constant current to the light emitter 3 to allow the light emitter 3 to emit light at a constant amount. A constant voltage power supply may be coupled in series with the light emitter 3 and a constant resistor. Otherwise, a constant current IC may be used. To save power, for example, power may be turned off during a measurement process, described later.

The controller 12 is configured to control the switch 15, as well as to determine paper characteristics based on a signal of the AD converter 13. In the present embodiment, a paper thickness is determined as one of the paper characteristics, for example. A specific determination method will be described later.

In addition to the characteristic regarding paper thicknesses of a thin paper, a plain paper, and a thick paper, for example, the paper characteristics include a characteristic regarding paper quality of a pure paper, a plain paper, a recycled paper, and a coated paper, for example. Depending on the paper characteristics, transmittance of a paper differs. Accordingly, a light-receiving output voltage differs. As a result, by measuring a light-receiving output voltage, paper characteristics can be detected.

The controller 12 is configured to control how a paper is fed, or to receive, from another controller (not illustrated), a trigger signal indicating that a paper is fed, for example, to identify the first state and the second state. The controller 12 includes a storage 12a and a calculator 12b. The controller 12 may be a microcomputer, for example.

The AD converter 13 includes a reference terminal 13a and a measurement voltage terminal 13b. The AD converter 13 is configured to measure a ratio between the voltage V1 of the measurement voltage terminal 13b and the voltage V0 of the reference terminal 13a. The AD converter 13 may be a 10-bit AD converter, for example. In this case, the AD converter 13 outputs a digital value (0 to 1023) representing a rank of the voltage V1, within a prescribed voltage range (0 to V0) divided into 2¹⁰=1024. When a microcomputer is used as the controller 12, for example/ an AD converter attached to the microcomputer may be used as the AD converter 13.

The storage circuit 14 is configured to hold an entered voltage as an analog voltage. The storage circuit 14 can be achieved by using a capacitative element, such as a capacitor, and a voltage follower.

The switch 15 can be controlled by the controller 12 for turning ON/OFF. For example, a metal-oxide-semiconductor field-effect transistor (MOSFET) may be used.

A switch IC may be used.

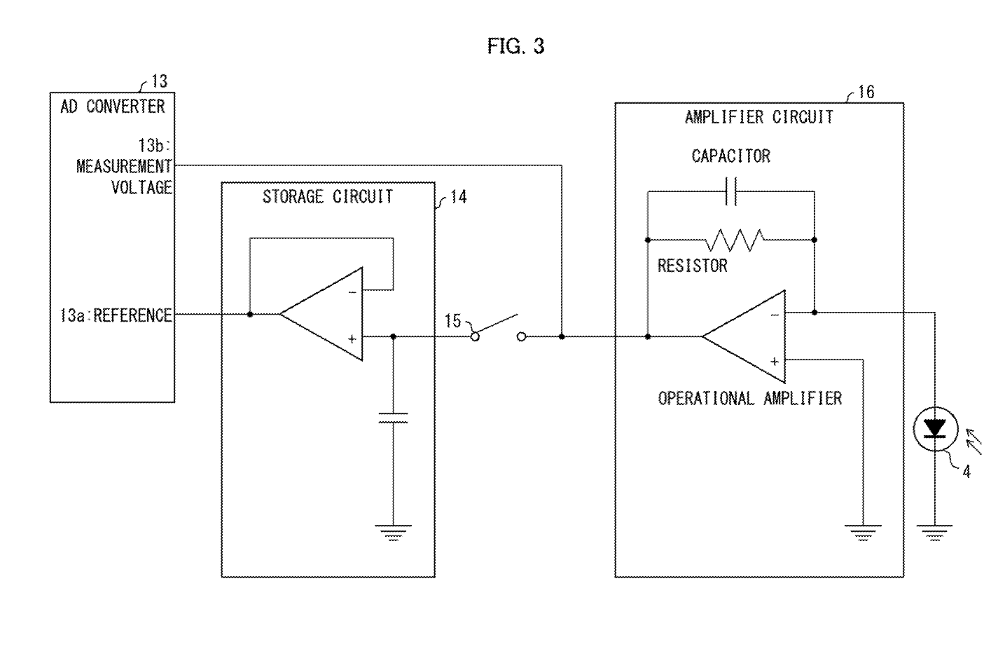

The amplifier circuit 16 is configured to convert a photocurrent from the photodetector 4 (photodiode) into a voltage proportional to the photocurrent, and to output the voltage to the measurement voltage terminal 13b of the AD converter 13 and the switch 15. As illustrated in FIG. 3, for example, the amplifier circuit 16 may be achieved with an operational amplifier coupled with a negative feedback resistor. FIG. 3 is a circuit diagram illustrating a coupling example and a configuration example of the AD converter 13, the storage circuit 14, the switch 15, and the amplifier circuit 16.

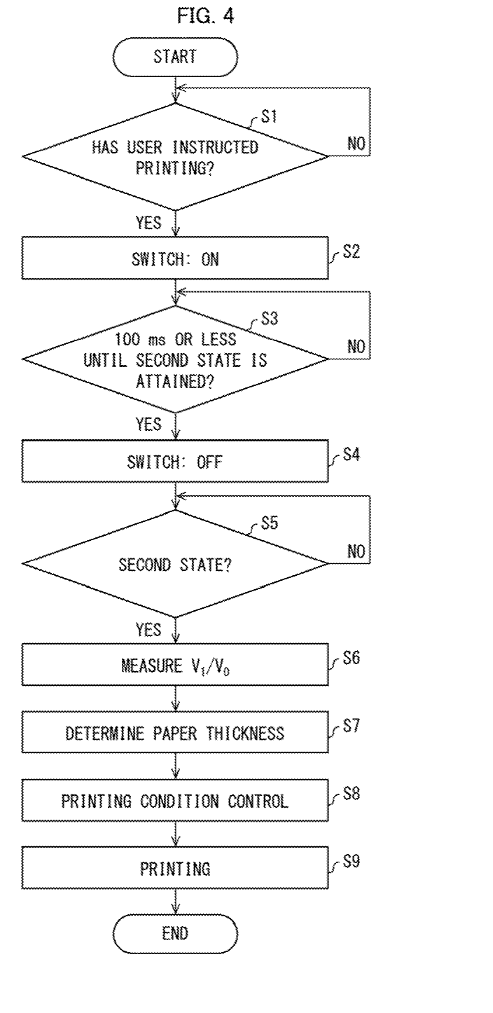

Next, a specific method for determining a paper thickness in the sensor device 1A having the configuration described above and a flow of printing in the linage forming apparatus will be described with reference to FIG. 4. FIG. 4 is a flowchart illustrating processing performed by the image forming apparatus including the sensor device 1A according to the present embodiment.

First, an instruction for printing by a user is waited (step S1). When an instruction for printing is given, the switch 15 is turned ON (step S2: first state). During the first state, the irradiated light L0 irradiated from the light emitter 3 is received by the photodetector 4. An output of the photodetector 4 is converted into a voltage by the amplifier circuit 16. The voltage is entered into the storage circuit 14. The storage circuit 14 holds this reference voltage (V0), and outputs the reference voltage V0 to the reference terminal 13a of the AD converter 13.

Next, the paper P to be fed is waited. Simultaneously, whether a time until the second state is attained is equal to or shorter than a prescribed time is determined (in the present embodiment, the prescribed time is, but not limited to, 100 ms) (step S3). While the paper P is approaching, at a timing when a time to attain the second state is 100 ms, the controller 12 turns OFF the switch 15 (step S4). Next, a timing when the second state is attained is waited (step S5). When the second state is attained, the photodetector 4 receives the transmitted light L1 transmitted through the paper P. The amplifier circuit 16 measures and outputs the voltage V1. At this time, in the AD converter 13, the reference terminal 13a is entered with the reference voltage V0 of the storage circuit 14. The measurement voltage terminal 13b is entered with the voltage V1 being measured. Upon the controller 12 gives an instruction, the AD converter 13 outputs a voltage ratio V1/V0 to the controller 12 with a prescribed bit count (step S6).

The storage 12a of the controller 12 is stored with a threshold value beforehand per the paper P that varies, based on measurement on the voltage ratio V1/V0. The data described above may be stored, as a database, in the storage 12a by a manufacturer of the sensor device 1A, for example. The calculator 12b compares the threshold value with the voltage ratio V1/V0 measured at this time to determine a paper thickness (step S7). For example, in a case where the AD converter 13 is a 10-bit AD converter, and a digital value ranging from 0 to 1023 inclusive is output, when a digital value fails within a range from 0 to 100 inclusive, it is determined that a thick paper is fed. When a digital value falls within a range from 101 to 300 inclusive, it is determined that a plain paper is fed. When a digital value falls within a range from 301 to 500 inclusive, it is determined that a thin paper is fed. Further, when a digital value falls within a range from 501 to 1023 inclusive, it is determined that the paper P is not fed (error, such as paper jamming).

Based on the determination, the image forming apparatus sets an image forming (printing) condition (step S8), and performs image forming (printing) onto the paper (step S9). Examples of image forming conditions (printing conditions) to be set by the controller 12 include a transfer current when toner is transferred onto the paper P, a feed speed for the paper P when toner is fused onto the paper P (fusing time), a temperature for a heating roller configured to pinch the paper P (fusing temperature), and pressure for a pressure roller (fusing pressure). For example, when the paper P is such a kind that has an uneven surface, the controller 12 increases the transfer current, and, further, increases the fusing pressure, compared with a case when the paper P is such a kind that has a flat surface. When the paper P is a thick paper, the controller 12 increases the fusing temperature or the fusing time, compared with a case when the paper P is a thin paper.

### Effects of the Disclosure

The sensor device 1A according to the present embodiment is capable of holding the reference voltage V0, during the first state, in the storage circuit 14 as an analog voltage to simultaneously enter the reference voltage V0 into the reference terminal 13a of the AD converter 13 and the voltage V1 measured during the second state into the measurement voltage terminal 13b of the AD converter 13.

As described above, the voltage ratio V1/V0 corresponding to transmittance of light is measured by fully utilizing a bit count of the AD converter 13. That is, the voltage ratio V1/V0 can be measured at higher precision, compared with a case when the reference voltage V0 and the voltage V1 being measured are respectively converted into the digital value D0 and the digital value D1, and then the digital ratio D1/D0 is calculated.

The sensor device 1A according to the present embodiment includes the switch 15 configured to switch whether an output voltage from the amplifier circuit 16 is to be entered into the storage circuit 14. Therefore, the switch 15 can switch whether an output voltage from the amplifier circuit 16 is to be entered into the storage circuit 14. As a result, an output voltage, during the first state, of the amplifier circuit 16 is entered, via the storage circuit 14, into the AD converter 13. An output voltage, during the second state, of the amplifier circuit 16 is directly entered into the AD converter 13.

As a result, the AD converter 13 is entered with both the output voltage, during the first state, and the output voltage, during the second state, of the amplifier circuit 16. Therefore, the AD converter 13 easily sets an output voltage of the storage circuit 14 as a reference voltage, and to convert, into a digital value, a voltage ratio between the reference voltage and an output voltage, during the second state, of the amplifier circuit 16.

The image forming apparatus according to the present embodiment includes the sensor device 1A according to the present embodiment, and is configured to set an image forming condition based on a result of measurement by the sensor device 1A. Therefore, a circuit configuration of the sensor device 1A can be simplified. The image forming apparatus capable of determining paper characteristics at higher precision, and of forming a high quality image can be thus achieved.

In the image forming apparatus according to the present embodiment, an image forming condition is at least one of a value of a voltage to be applied to a transfer device and a value of a current to be supplied to the transfer device, pressure to be applied to a paper by a fuser, a temperature of heating for the paper by the fuser, and a speed of feeding for the paper by the fuser. Therefore, with various image forming conditions described above, the image forming apparatus capable of forming a high quality image can be achieved.

### Modification

In the example described above, the irradiated light L0 enters into the photodetector 4 during the first state. However, an optical element may lie between the light emitter 3 and the photodetector 4 during the first state. When a difference in amount of light to be received by the photodetector 4 between the first state and the second state reduces, precision in measurement improves. Its effects are compatible with the effects of the present disclosure.

### Second Embodiment

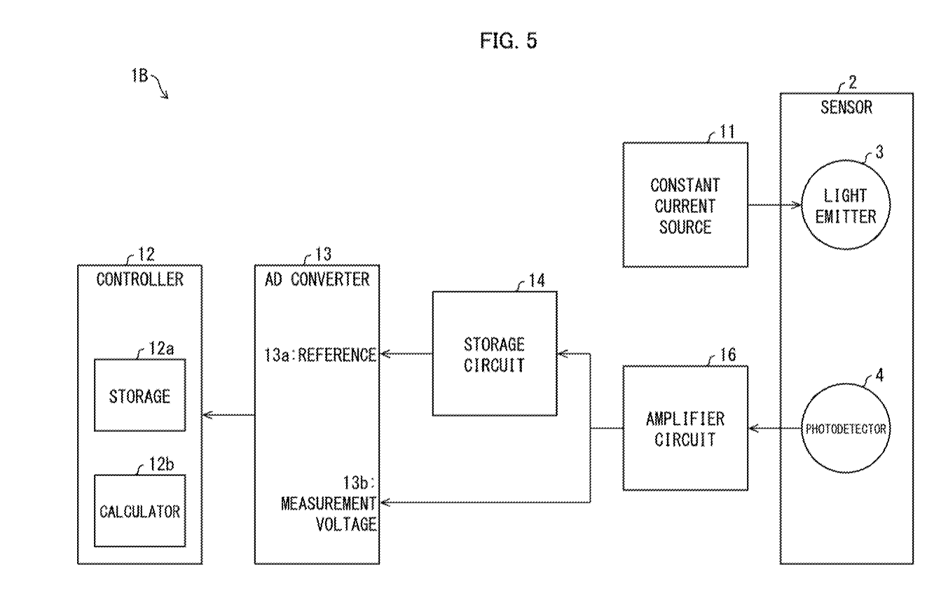

Another embodiment of the present disclosure will be described herein with reference to FIGS. 5 to 8. Note that, for convenience of explanation, components illustrated in respective embodiments are designated by the same reference numerals as those having the same function, and the descriptions of these components will be omitted.

FIG. 5 is a block diagram illustrating a configuration of a main part of a sensor device 1B. In the present embodiment, the switch 15 is not provided, different from the sensor device 1A according to the first embodiment.

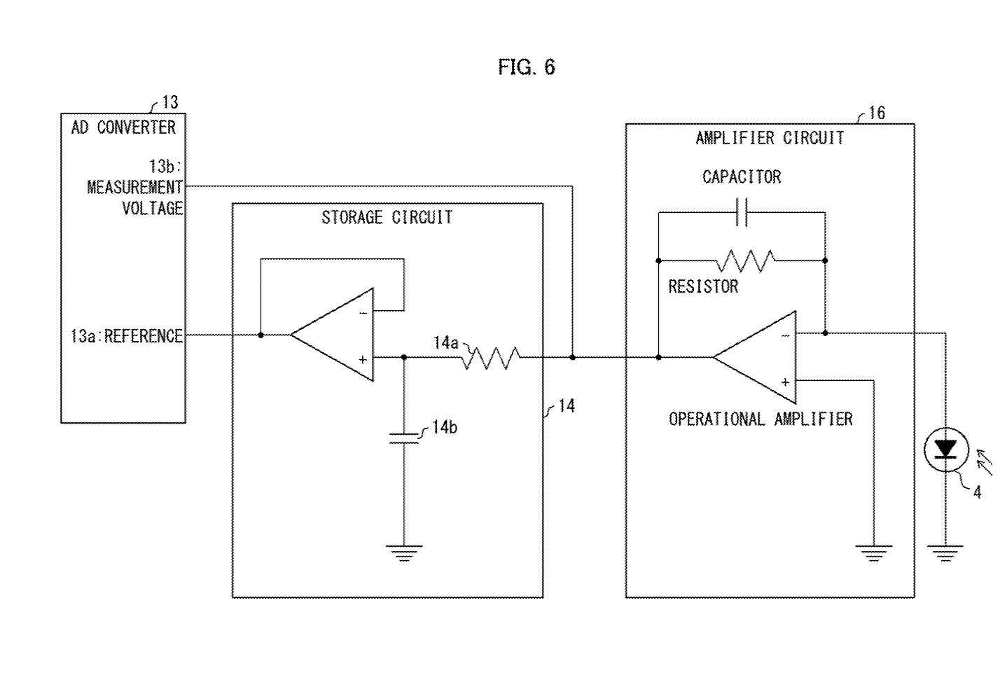

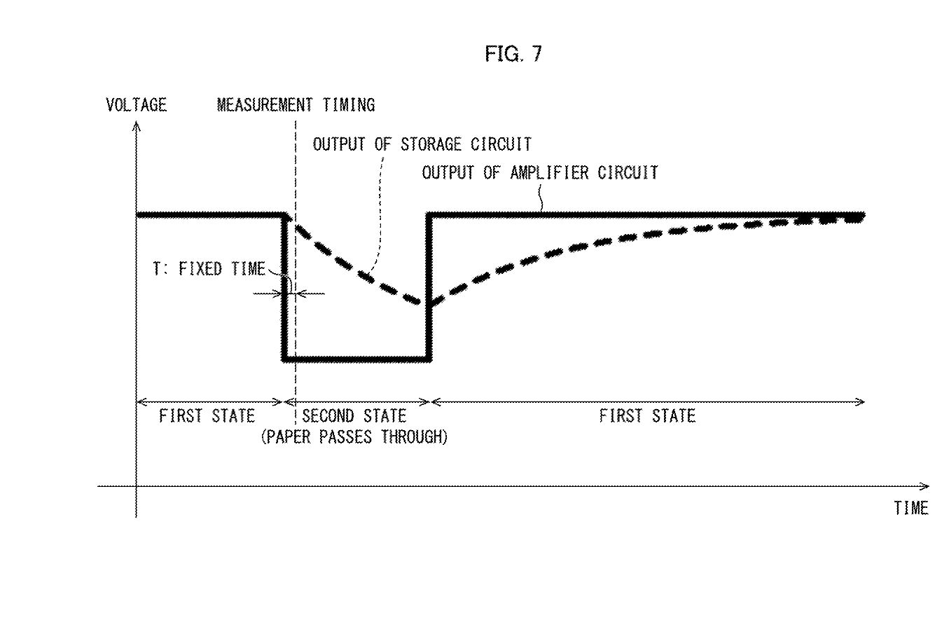

A configuration of the sensor device 1B according to the present embodiment will be described with reference to FIGS. 5 to 7. FIG. 6 is a circuit diagram illustrating the configuration of the main part of the sensor device 1B. FIG. 7 is a graph illustrating an output of the amplifier circuit 16 and an output of the storage circuit 14, according to the present embodiment.

As illustrated in FIG. 6, in the sensor device 1B according to the present embodiment, the AD converter 13, the storage circuit 14, and the amplifier circuit 16 are provided. However, the switch 15 provided in the sensor device 1A according to the first embodiment is not provided. The storage circuit 14 is provided with a resistor 14a on its input side.

As can be seen from the circuit diagram of the coupling example and the configuration example, the storage circuit 14 is entered with an output, during the first state, of the amplifier circuit 16. The storage circuit 14 is further entered with an output, during the second state, of the amplifier circuit 16.

As a result, as illustrated in FIG. 7, an input voltage of the storage circuit 14, i.e., an output voltage of the amplifier circuit 16, can be output as a signal obtained by integrating the voltage with a certain time constant τ (determined by a resistance value of the resistor 14a and a capacitance of the capacitor 14b).

Specifically, when the paper P passes through the sensor 2, in FIG. 7, the first state turns to the second state. Further, after that, when no paper is detected, the first state resumes. An output voltage of the amplifier circuit 16 is proportional to a current to be output by the photodetector 4 within a time approximately equal to a time constant (in here, fully shorter) of the amplifier circuit 16, i.e., is lower than an output voltage during the first state, during a time when the second state is attained. On the other hand, an output voltage of the storage circuit 14 follows an output voltage of the amplifier circuit 16 with a delay approximately equal to the time constant τ. Herein, a time required by the paper P to pass through is, but not limited to, one second. The time constant τ of the storage circuit 14 is, but not limited to, also one second. The time constant of the amplifier circuit 16 is, but not limited to, 0.1 ms. When an input voltage of the storage circuit 14, i.e., the voltage V1 output by the amplifier circuit 16, changes during a time t=0 from an output Va to an output Vb, the voltage V0 output, by the storage circuit 14 is obtained through (Formula 1) described below.

Expression 1

V₀=V_{b}+(Vₐ−V_{b})exp(−t/τ) (Formula 1)

In (Formula 1), when the time t=τ/100, e x p (−0.01)≈0.99. Therefore, V0≈0.99 Va+0.01 Vb. The voltage V0 being output is equal to the output Va at a precision of 1%. Therefore, when the time t=τ/100, an output of the AD converter 13 is one that is obtained by measuring the voltage ratio V1/V0 at a precision of 1%.

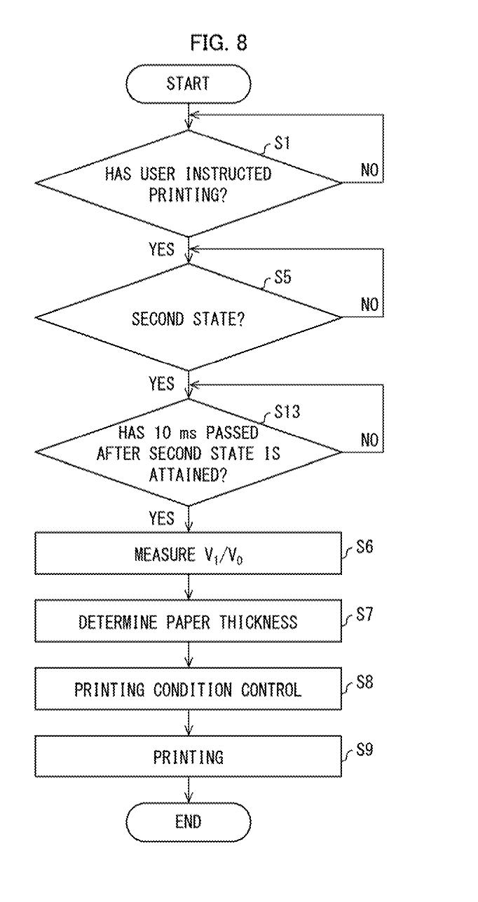

FIG. 8 is a flowchart illustrating processing performed by an image forming apparatus including the sensor device 1B according to the present embodiment. In FIG. 8, steps S2 to S4 are not provided. Instead, step S13 is provided, different from the flowchart in FIG. 4 according to the first embodiment.

As illustrated in FIG. 8, in step S1, when a user gives an instruction for printing, the second state is waited (step S5). A fixed time T (in here, 10 ms) is also waited (step S13). That is, for the fixed time T, the time constant x representing a delay when an output voltage of the storage circuit 14 follows an output voltage of the amplifier circuit 16, and precision of a voltage ratio are taken into account. It is preferable that the fixed time T be approximately one hundredth of the time constant σ (=one second) of the storage circuit 14, for example. It is required that the fixed time T be fully longer than the time constant of the amplifier circuit 16, i.e., be fully longer than 0.1 ms.

After that, similar to the first embodiment, the voltage ratio V1/V0 is measured. A paper thickness is determined. A printing condition control takes place. Printing takes place (steps S6 to S9).

### Modification

In the above description, the controller 12 is configured to control how a paper is fed, or to receive, from another controller (not illustrated), a trigger signal indicating that a paper is fed, for example, to identify the first state and the second state to measure the voltage ratio V1/V0 after 10 ms when the second state is attained. However, the present disclosure is not limited to the above description. For example, the voltage ratio V1/V0 may be measured. A decrease in the voltage ratio V1/V0 when the second state is attained may then be detected. A shift from the first state to the second state may thus be recognized.

As described above, in addition to determining a thickness of a paper at higher precision, when the paper P is reached can be detected.

As described above, the sensor device 1B according to the present embodiment enters an output voltage, during the first state, of the amplifier circuit 16 into the storage circuit 14. Even during the second state, the storage circuit 14 holds the output voltage, during the first state, of the amplifier circuit 16. During the second state, the AD converter 13 sets an output voltage, during the first state, of the storage circuit 14 as a reference voltage, and converts, into a digital value, a voltage ratio between the reference voltage and an output voltage, during the second state, of the amplifier circuit 16.

In the present embodiment, a state is attained, where the switch 15 according to the first embodiment is kept turned on or the switch 15 according to the first embodiment is not provided.

In this case, until the fixed time T passes after the first state turns to the second state, the amplifier circuit 16 and the storage circuit 14 do not operate normally. The AD converter 13 cannot thus output a highly precise voltage ratio.

In the present embodiment, during the second state, the AD converter 13 sets an output voltage, during the first state, of the storage circuit 14 as a reference voltage, and converts, into a digital value, a voltage ratio between the reference voltage and an output voltage, during the second state, of the amplifier circuit 16. As a result, a highly precise digital value of a voltage ratio can be obtained.

### Third Embodiment

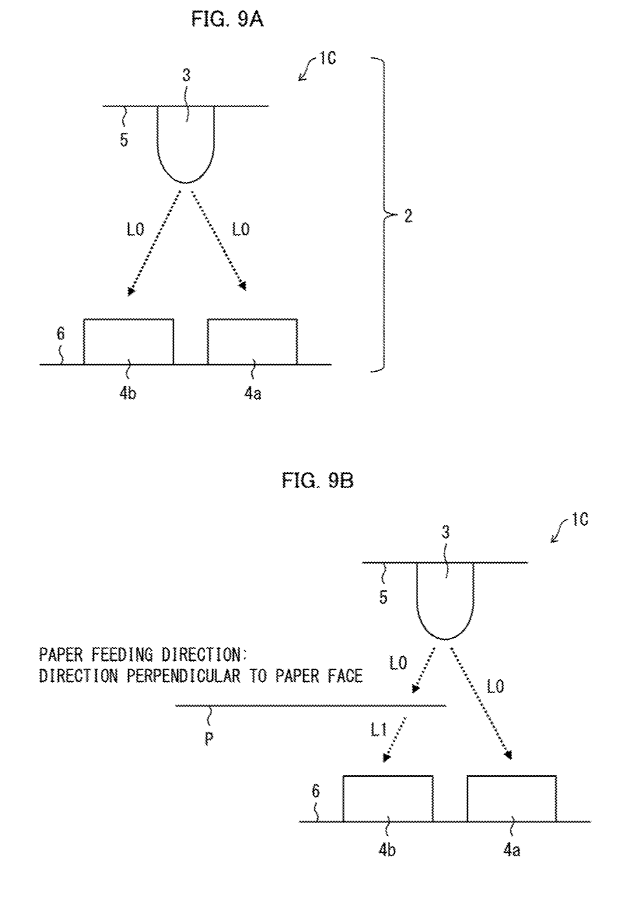

Still another embodiment, of the present disclosure will be described with reference to FIGS. 9 to 12. Note that, for convenience of explanation, components illustrated in the embodiment are designated by the same reference numerals in the first and second embodiments described above as those having the same function, and the descriptions of these components will be omitted.

FIG. 9A is a view illustrating the first state of the sensor 2 of a sensor device 1C according to the present, embodiment. FIG. 9B is a view illustrating the second state. As illustrated in FIG. 9A, the sensor 2 of the sensor device 1C according to the present embodiment includes two photodetectors 4a and 4b in a direction perpendicular to a feeding direction of the paper P, different from the first and second embodiments.

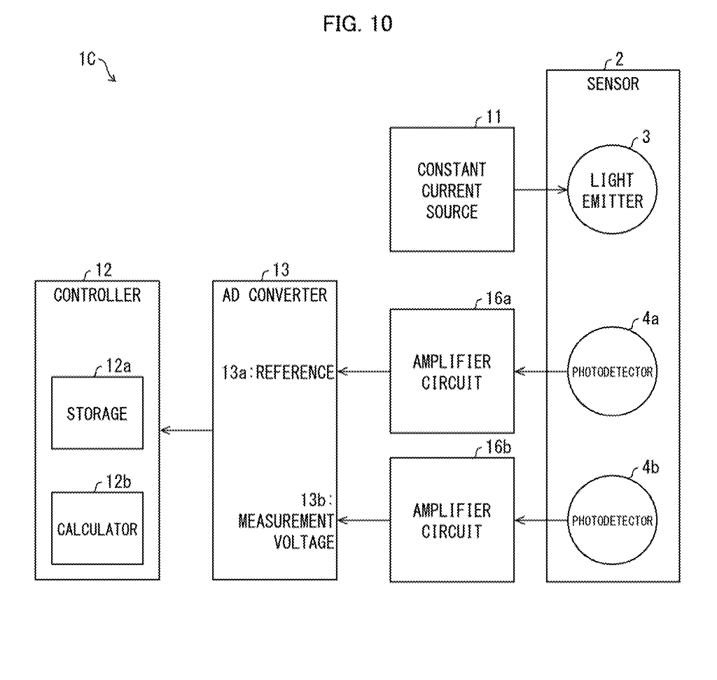

A configuration of the sensor device 1C according to the present embodiment will be described with reference to FIGS. 9 and 10. FIG. 10 is a block diagram illustrating a configuration of a main part of the sensor device 1C.

As illustrated in FIGS. 9A and 9B, in the sensor device 1C according to the present embodiment, the light emitter 3 irradiates the irradiated light L0 at a wider angle. As the light emitter 3, an LED configured to irradiate light at a wider angle may be used. A lens and a light guide, for example, may be used to expand light. As illustrated in FIG. 9A, during the first state, the irradiated light L0 enters into the photodetector 4a serving as a first photodetector and the photodetector 4b serving as a second photodetector. It is preferable that the photodetectors 4a and 4b be symmetry in position with respect to the light emitter 3. In other words, it is preferable that amounts of light to enter into the two photodetectors 4a and 4b be identical. As illustrated in FIG. 9B, during the second state, some of the irradiated light L0 enters into the photodetector 4a serving as the first photodetector. The rest of the irradiated light L0 is absorbed by the paper P, and scatters and enters, as the transmitted light L1, into the photodetector 4b serving as the second photodetector.

As illustrated in FIG. 10, the sensor device 1C according to the present embodiment includes a plurality of amplifier circuits, i.e., an amplifier circuit 16a serving as a first amplifier circuit and an amplifier circuit 16b serving as a second amplifier circuit, respectively corresponding to the photodetector 4a serving as the first photodetector and the photodetector 4b serving as the second photodetector, different from the first and second embodiments. By taking into account individual differences in sensitivity when the photodetectors 4a and 4b are respectively procured at greater quantities to mass-produce the sensor device 1C, amplification factors of the amplifier circuits 16a and 16b being set allow an output V0b of the amplifier circuit 16b to be always smaller than an output. V0a of the amplifier circuit 16a during the first state. For example, when individual differences in sensitivity when the photodetectors 4a and 4b are respectively procured at greater quantities are each approximately 5%, a ratio of sensitivity between the photodetector 4a and the photodetector 4b reaches 10% in maximum. Therefore, by specifying an amplification factor of the amplifier circuit 16b to approximately 80% of an amplification factor of the amplifier circuit 16a, the output V0b does not exceed the output V0a even an individual difference in sensitivity arises.

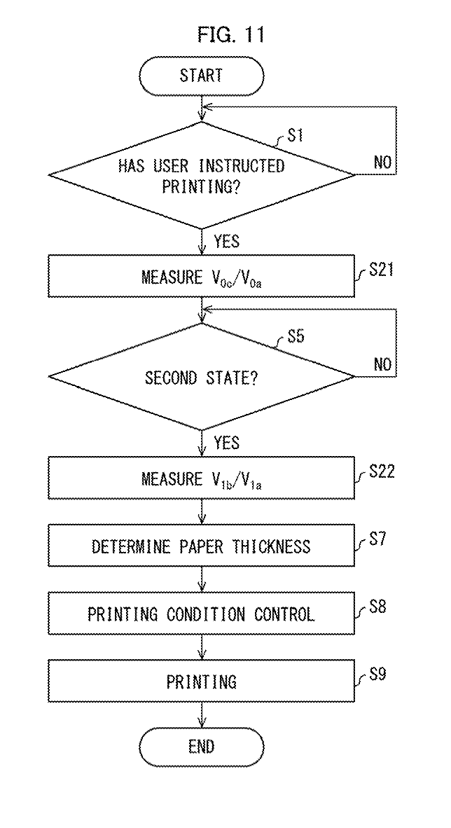

FIG. 11 is a flowchart illustrating processing performed by an image forming apparatus including the sensor device 1C according to the present embodiment.

As illustrated in FIG. 11, after a user gives an instruction for printing (step S1), a voltage ratio V0b/V0a is measured during the first state (step S21). Next, attaining the second state is waited (step S5). During the second state, a voltage ratio V1b/V1a between an output V1b of the amplifier circuit 16b and an output V1a of the amplifier circuit 16a is measured (step S22).

Next, by using a result of (Formula 2) described below, and by using a method identical to the method in the first embodiment, a paper thickness is determined. Printing conditions are controlled. Printing takes place (steps S1 to S9).

$$\begin{matrix}[ Expression 2 ] & \\ \frac{{V}_{1}}{{V}_{0}} = \frac{{V}_{1 b}}{{V}_{1 a}} \times \frac{{V}_{0 a}}{{V}_{0 b }} & ( Formula 2 )\end{matrix}$$

As described above, in steps S21 and S22, even when a power supply voltage fluctuates, for example, and an amount of light emitted from the light emitter 3 changes, the voltage ratio V1/V0 can be calculated at higher precision.

The controller 12 may be configured to control how a paper is fed, or to receive, from another controller (not illustrated), a trigger signal indicating that a paper is fed, for example, to identify the first, state and the second state. Otherwise, the controller 12 may detect a decrease in output voltage of the amplifier circuit 16b to identify the first state and the second state.

### Modification

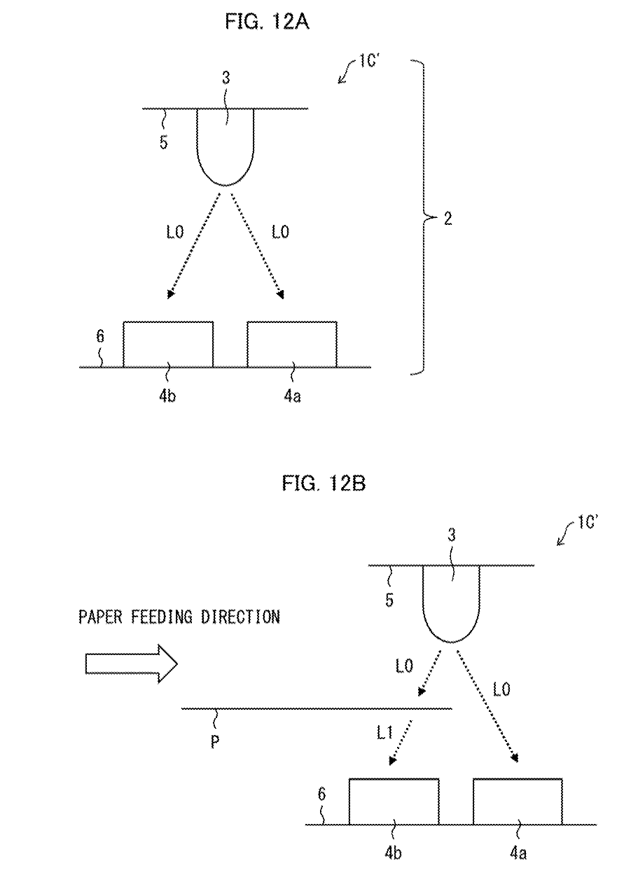

For example, as illustrated in FIG. 12A, the sensor 2 of a sensor device 1C′ may include the two photodetectors 4a and 4b in the feeding direction of the paper P. FIG. 12A is a view illustrating the first state of the sensor 2 according to a modification. FIG. 12B is a view illustrating the second state.

In the present modification, as illustrated in FIG. 12B, a state when the paper P lies between the light emitter 3 and the photodetector 4b, but does not lie between the light emitter 3 and the photodetector 4a is referred to as the second state.

As described above, the modification is available when a position of an end of the paper P differs due to a size of the paper P.

As described above, the sensor devices 1C and 1C′ according to the present embodiment each include the light emitter 3 configured to irradiate light, the photodetector 4a configured to serve as the first photodetector to receive irradiated light from the light emitter 3, the photodetector 4b configured to serve as the second photodetector to receive transmitted light from a paper, among the irradiated light from the light emitter 3, the amplifier circuit 16a configured to serve as the first amplifier circuit to convert, an output of the photodetector 4a into a voltage, the amplifier circuit 16b configured to serve as the second amplifier circuit to convert an output of the photodetector 4b into a voltage, and the AD converter 13 configured to set an output voltage of the amplifier circuit 16a as a reference voltage, and to convert, into a digital value, a voltage ratio between the reference voltage and an output voltage of the amplifier circuit 16b. Based on an output from the AD converter 13, paper characteristics are detected.

According to the configuration, even when a power supply voltage fluctuates, for example, and an amount of light emitted from the light emitter 3 changes, a voltage ratio and its digital value can be calculated at higher precision.

### Fourth Embodiment

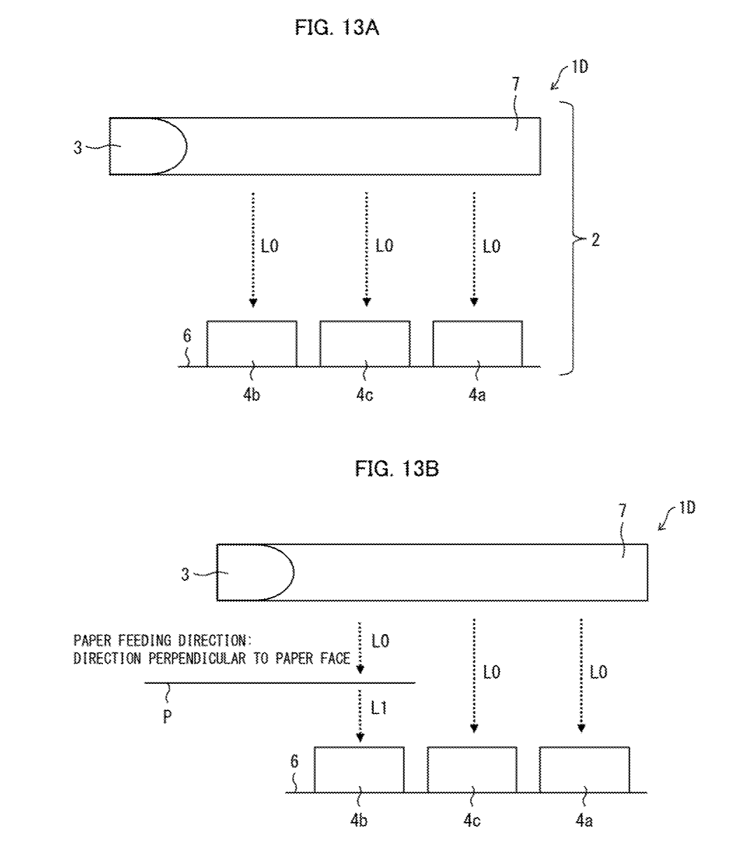

Still another embodiment of the present disclosure will be described with reference to FIGS. 13 to 15. Note that, for convenience of explanation, components illustrated in the embodiment are designated by the same reference numerals in the first to third embodiments described above as those having the same function, and the descriptions of these components will be omitted.

FIG. 13A is a view illustrating the first state of the sensor 2 of a sensor device 1D according to the present embodiment.

FIG. 13B is a view illustrating the second state.

As illustrated in FIG. 13A, the sensor 2 of the sensor device 1D according to the present embodiment includes a light guide 7 configured to linearly irradiate light from the light emitter 3, and three photodetectors, i.e., the photodetectors 4a and 4b, and a photodetector 4c, in a direction perpendicular to the feeding direction of the paper P, different from the first to third embodiments.

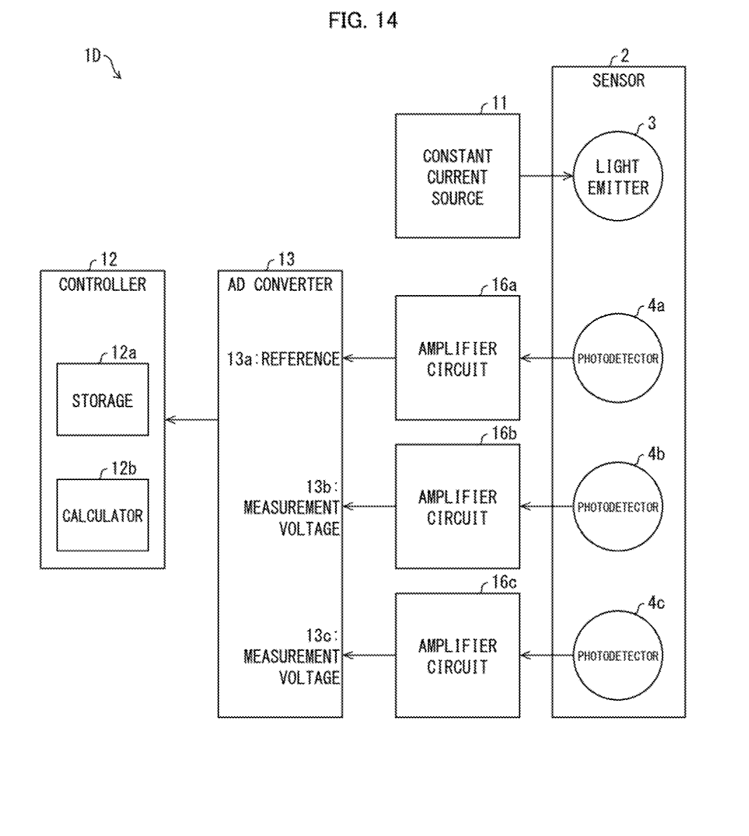

A configuration of the sensor device 1D according to the present embodiment will be described with reference to FIGS. 13A and 13B and FIG. 14. FIG. 14 is a block diagram illustrating a configuration of a main part of the sensor device 1D.

As illustrated in FIG. 13A, in the sensor device 1D according to the present embodiment, the three photodetectors, i.e., the photodetector 4a serving as the first photodetector, the photodetector 4c serving as a third photodetector, and the photodetector 4b serving as the second photodetector, are arranged in order in the direction perpendicular to the feeding direction of the paper P.

As a result, during the first state, the irradiated light L0 enters into the photodetector 4a serving as the first photodetector, the photodetector 4c serving as the third photodetector, and the photodetector 4b serving as the second photodetector. It is preferable that the light guide 7 allows light to expand linearly and evenly. As illustrated in FIG. 13B, during the second state, some of the irradiated light L0 enters into the photodetector 4a serving as the first photodetector. The rest of the irradiated light L0 is absorbed by the paper P, and scatters and turns to the transmitted light L1 to enter into the photodetector 4b serving as the second photodetector. Depending on the size of the paper P, the irradiated light L0 or the transmitted light L1 may enter into the photodetector 4c serving as the third photodetector.

As illustrated in FIG. 14, the present embodiment includes the amplifier circuit 16a serving as the first, amplifier circuit, an amplifier circuit 16c serving as a third amplifier circuit, and the amplifier circuit 16b serving as the second amplifier circuit, respectively corresponding to the photodetector 4a serving as the first photodetector, the photodetector 4c serving as the third photodetector, and the photodetector 4b serving as the second photodetector, different from the first to third embodiments. However, similar to the third embodiment, amplification factors of the amplifier circuits 16a, 16b, and 16c being set allow the output V0b of the amplifier circuit 16b and an output V0c of the amplifier circuit 16c to be always smaller than the output V0a of the amplifier circuit 16a during the first state. The AD converter 13 is provided with a measurement voltage terminal 13c configured to receive an output V1c from the amplifier circuit 16c.

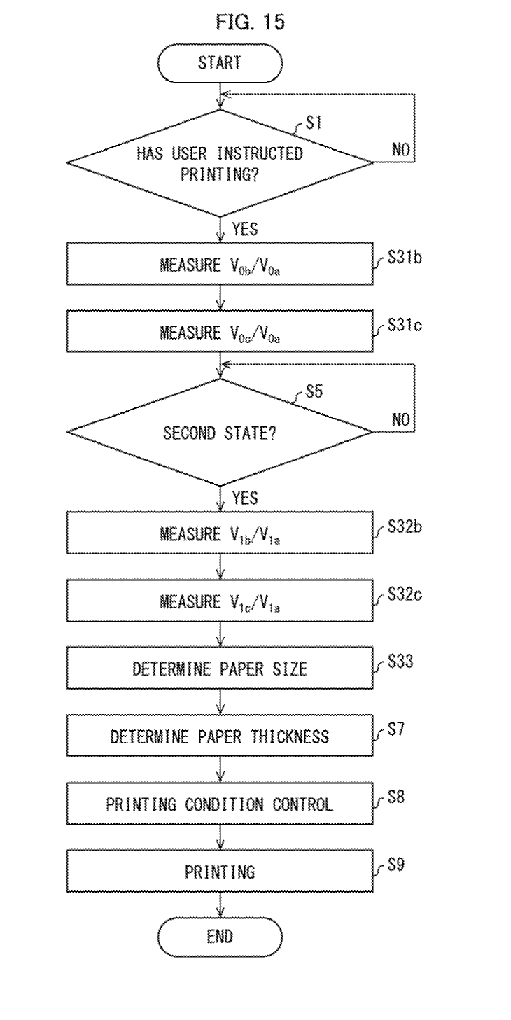

FIG. 15 is a flowchart illustrating processing performed by an image forming apparatus including the sensor device 1D according to the present embodiment. As illustrated in FIG. 15, after a user gives an instruction for printing (step S1), the voltage ratio V0b/V0a and a voltage ratio V0c/V0a are measured during the first state (steps S31b and S31c). Next, attaining the second state is waited (step S5). During the second state, the voltage ratio V1b/V1a between the output V1b of the amplifier circuit 16b and the output V1a of the amplifier circuit 16a, and a voltage ratio V1c/V1a between the output V1c of the amplifier circuit 16c and the output V1a of the amplifier circuit 16a are measured (steps S32b and S32c). Next, the voltage ratio V1c/V1a is compared with a certain threshold value. When the voltage ratio V1c/V1a is smaller than the certain threshold value, it is determined that the paper P is present over the photodetector 4c, as well as, it is determined that the size of the paper P is greater. When the voltage ratio V1c/V1a is greater than the certain threshold value, it is determined that the paper P is not present over the photodetector 4c, as well as it is determined that the size of the paper P is smaller (step S33).

In here, the photodetector 4c configured to serve as the third photodetector to detect a change in amount of light depending on the size of the paper P is solely provided. However, the present disclosure is not limited to the embodiment. By increasing photodetectors in number, the size of the paper P can be precisely determined.

For a thickness of the paper P, by using a result of (Formula 3) described below, and by using a method identical to the method in the first embodiment, a paper thickness is determined. Printing conditions are controlled. Printing takes place.

$$\begin{matrix}[ Expression 3 ] & \\ \frac{{V}_{1}}{{V}_{0}} = \frac{{V}_{1 c}}{{V}_{1 a}} \times \frac{{V}_{0 a}}{{V}_{0 c }} & ( Formula 3 )\end{matrix}$$

Otherwise, when the paper P is present over the photodetector 4b, (Formula 4) may be used to average data of the photodetector 4b and the photodetector 4c.

$$\begin{matrix}[ Expression 4 ] & \\ \frac{{V}_{1}}{{V}_{0}} = \frac{1}{2} ( \frac{{V}_{1 b}}{{V}_{1 a}} \times \frac{{V}_{0 a}}{{V}_{0 b}} + \frac{{V}_{1 c}}{{V}_{1 a}} \times \frac{{V}_{0 a}}{{V}_{0 c}} ) & ( Formula 4 )\end{matrix}$$

As described above, differences in thickness within a single paper can be averaged, allowing a highly precise determination.

The controller 12 may be configured to control how a paper is fed, or to receive, from another controller (not illustrated), a trigger signal indicating that a paper is fed, for example, to identify the first state and the second state. Otherwise, the controller 12 may detect a decrease in output voltage of the amplifier circuit 16c to identify the first state and the second state.

As described above, in addition to the photodetector 4a serving as the first photodetector and the photodetector 4b serving as the second photodetector, it is preferable that the sensor device ID according to the present embodiment include the photodetector 4c serving as at least one third photodetector to determine a paper size in addition to determining a paper thickness.

Therefore, for example, by providing the photodetector 4c configured to detect whether a paper having a greater size passes through, it can be determined that, when the photodetector 4c detects transmitted light from a paper, the paper having a greater size has passed. It can be determined that, when the photodetector 4c does not detect transmitted light from a paper, a paper having a greater size has not yet passed.

By providing a plurality of the photodetectors 4c, a plurality of kinds of paper sizes can be determined.

### SUMMARY

The sensor device according to a first aspect of the present disclosure includes a light emitter configured to irradiate light, a photodetector configured to receive, during a first state where no paper exists, irradiated light from the light emitter, and to receive, during a second state where a paper exists, transmitted light from the paper, an amplifier circuit configured to convert an output of the photodetector into a voltage, a storage circuit configured to hold an output voltage, during the first state, from the amplifier circuit, and an AD converter configured to set an output voltage of the storage circuit as a reference voltage, and to convert, into a digital value, a voltage ratio between the reference voltage and an output voltage, during the second state, of the amplifier circuit. Based on an output from the AD converter, paper characteristics are detected.

According to the configuration, an output voltage, during the first state, of the amplifier circuit is held in the storage circuit. The AD converter is entered with the output voltage, during the first state, held in the storage circuit, and an output, voltage, during the second state, of the amplifier circuit. The AD converter sets the output voltage, during the first state, held in the storage circuit, as a reference voltage, obtains a voltage ratio between the reference voltage and an output voltage, during the second state, of the amplifier circuit, and converts the voltage ratio into a digital value.

As a result, a digital value of a voltage ratio can be calculated at higher precision, compared with a case when a reference voltage during the first state and a measurement voltage during the second state are respectively converted into digital values, and then a digital ratio is calculated.

Therefore, a circuit configuration of a sensor device can be simplified. The sensor device capable of determining paper characteristics at higher precision, and of forming a high quality image can be thus achieved.

A sensor device according to a second aspect of the present disclosure may include a switch configured to switch whether an output voltage from the amplifier circuit is to be entered into the storage circuit.

Therefore, an output voltage, during the first state, of the amplifier circuit is entered, via the storage circuit, into the AD converter. An output voltage, during the second state, of the amplifier circuit is also entered into the AD converter. Therefore, the AD converter can easily set an output voltage of the storage circuit as a reference voltage, and car easily convert, into a digital value, a voltage ratio between the reference voltage and an output voltage, during the second state, of the amplifier circuit.

In a sensor device according to a third aspect of the present disclosure, an output voltage, during the first state, from the amplifier circuit can be entered into the storage circuit. Even during the second state, the storage circuit can hold the output voltage, during the first state, from the amplifier circuit. During the second state, the AD converter can set an output voltage, during the first state, of the storage circuit as a reference voltage, and can convert, into a digital value, a voltage ratio between the reference voltage and an output voltage, during the second state, of the amplifier circuit. As a result, a highly precise digital value of a voltage ratio can be obtained.

A sensor device according to a fourth aspect of the present disclosure includes a light emitter configured to irradiate light, a first photodetector configured to receive irradiated light from the light emitter, a second photodetector configured to receive transmitted light from a paper, among the irradiated light from the light emitter, a first amplifier circuit configured to convert an output of the first photodetector into a voltage, a second amplifier circuit configured to convert an output of the second photodetector into a voltage, and an AD converter configured to set an output voltage of the first amplifier circuit as a reference voltage, and to convert, into a digital value, a voltage ratio between the reference voltage and an output voltage of the second amplifier circuit. Based on an output from the AD converter, paper characteristics are detected.

According to the configuration, even when a power supply voltage fluctuates, for example, and an amount of light emitted from the light emitter changes, a voltage ratio and its digital value can be calculated at higher precision.

In a sensor device according to a fifth aspect of the present disclosure, it is preferable that, in addition to the first photodetector and the second photodetector, at least one third photodetector be included to determine a paper size in addition to determining the paper characteristics.

Therefore, for example, by providing the third photodetector configured to detect whether a paper having a greater size passes through, it can be determined that, when the third photodetector detects transmitted light from a paper, the paper having a greater size has passed. It can be determined that, when the third photodetector does not detect transmitted light from a paper, a paper having a greater size has not yet passed.

By providing a plurality of the third photodetectors, a plurality of kinds of paper sizes can be determined.

An image forming apparatus according to a sixth aspect of the present disclosure includes one of the sensor devices described above, and is configured to set an image forming condition based on a result of measurement by the sensor device.

According to the configuration, a circuit configuration of the sensor device can be simplified. The image forming apparatus capable of determining paper characteristics at higher precision, and of forming a high quality image can be thus achieved.

In an image forming apparatus according to a seventh aspect of the present disclosure, the image forming condition can be at least one of a value of a voltage to be applied to a transfer device
and a value of a current to be supplied to the transfer device, pressure to be applied to the paper by a fuser, a temperature of heating for the paper by the fuser, and a speed of feeding for the paper by the fuser.

Therefore, with various image forming conditions described above, the image forming apparatus capable of forming a high quality image can be achieved.

The present disclosure is not limited to each of the above-described embodiments. It is possible to make various modifications within the scope of the claims. An embodiment obtained by appropriately combining technical elements each disclosed in different embodiments falls also within the technical scope of the present disclosure. Furthermore, technical elements disclosed in the respective embodiments may be combined to provide a new technical feature.

While preferred embodiments of the present invention have been described above, it is to be understood that variations and modifications will be apparent to those skilled in the art without departing from the scope and spirit of the present invention. The scope of the present invention, therefore, is to be determined solely by the following claims.

1. A sensor device comprising:
a light emitter configured to irradiate light;
a photodetector configured to receive, during a first state where no paper exists, irradiated light from the light emitter, and to receive, during a second state where a paper exists, transmitted light from the paper;
an amplifier circuit configured to convert an output of the photodetector into a voltage;
a storage circuit configured to hold an output voltage, during the first state, from the amplifier circuit; and
an AD converter configured to set an output voltage of the storage circuit as a reference voltage, and to convert, into a digital value, a voltage ratio between the reference voltage and an output voltage, during the second state, of the amplifier circuit,
wherein, based on an output from the AD converter, paper characteristics are detected.

2. The sensor device according to claim 1, further comprising a switch configured to switch whether an output voltage from the amplifier circuit is to be entered into the storage circuit.

3. The sensor device according to claim 1,
wherein an output voltage, during the first state, from the amplifier circuit is entered into the storage circuit,
even during the second state, the storage circuit holds the output voltage, during the first state, from the amplifier circuit, and
during the second state, the AD converter sets an output voltage, during the first state, of the storage circuit as a reference voltage, and converts, into a digital value, a voltage ratio between the reference voltage and an output voltage, during the second state, of the amplifier circuit.

4. A sensor device comprising:
a light emitter configured to irradiate light;
a first photodetector configured to receive irradiated light from the light emitter;
a second photodetector configured to receive transmitted light from a paper, among the irradiated light from the light emitter;
a first amplifier circuit configured to convert an output of the first photodetector into a voltage;
a second amplifier circuit configured to convert an output of the second photodetector into a voltage; and
an AD converter configured to set an output voltage of the first amplifier circuit as a reference voltage, and to convert, into a digital value, a voltage ratio between the reference voltage and an output voltage of the second amplifier circuit,
wherein, based on an output from the AD converter, paper characteristics are detected.

5. The sensor device according to claim 4,
further comprising, in addition to the first photodetector and the second photodetector, at least one third photodetector,
wherein, in addition to a determination of the paper characteristics, a paper size is determined.

6. An image forming apparatus comprising the sensor device according to claim 1,
wherein an image forming condition is set based on a result of measurement by the sensor device.

7. The image forming apparatus according to claim 6,
wherein the image forming condition is at least one of a value of a voltage to be applied to a transfer device and a value of a current to be supplied to the transfer device, pressure to be applied to the paper by a fuser, a temperature of heating for the paper by the fuser, and a speed of feeding for the paper by the fuser.

8. An image forming apparatus comprising the sensor device according to claim 4,
wherein an image forming condition is set based on a result of measurement by the sensor device.

9. The image forming apparatus according to claim 8,
wherein the image forming condition is at least one of a value of a voltage to be applied to a transfer device and a value of a current to be supplied to the transfer device, pressure to be applied to the paper by a fuser, a temperature of heating for the paper by the fuser, and a speed of feeding for the paper by the fuser.
